(19) United States
(12) Patent Application Publication
(10) Pub. No.: US 2006/0052880 A1
(43) Pub. Date: Mar. 9, 2006

(54) Plasma sprayed porous coating for medical implants

(75) Inventors: Robert E. Brosnahan III, Germantown, TN (US); Randy Fesmire, Hernando, MS (US); Harsh Gupta, Memphis, TN (US); Daniel A. Heuer, Memphis, TN (US); Gordon Hunter, Memphis, TN (US); Vivek Pawar, Memphis, TN (US)

Correspondence Address: SMITH & NEPHEW, INC., 1450 E. BROOKS ROAD, MEMPHIS, TN, 38116, US

(73) Assignee: Smith & Nephew, Inc.

(21) Appl. No.: 10/937,147

(22) Filed: Sep. 9, 2004

(51) Int. Cl. A61F 2/30; A61F 2/28; B05D 1/02

(52) U.S. Cl. 623/23.39; 623/23.5; 623/23.51; 427/2.27

(57) ABSTRACT

A prosthesis having an oxidized zirconium surface and a porous textured surface is described. A major advantage of the prosthesis is increased service life. Additionally, a process of manufacturing the prosthesis is described.

### TECHNICAL FIELD

The invention related to medical implants comprising a surface layer of oxidized zirconium and which also include a metallic porous coating disposed on at least a portion of the exterior of the structural member.

### BACKGROUND OF THE INVENTION

For a variety of reasons, it is sometimes necessary to surgically correct an earlier implanted medical implant (most commonly a prosthetic joint) or replace it with an entirely new medical implant. Typically, this results from either a loosening of the implant in the implant site, or the deterioration of the implant due to forces such as abrasion. Ideally, a medical implant is often formed from a high-strength material which is not only able to accommodate the various loading conditions that it may encounter, but is also non-toxic to, and otherwise biocompatible with, the human body. It is also preferable to implant the device in such a way as to enhance fixation over the long term.

A number of advances have been made to increase service life of medical implants by increasing their resistance to forces such as abrasion. The advent of oxidized zirconium, first described by Davidson in U.S. Pat. No. 5,037,438 has provided a surface with superior hardness which is also resistance to brittle fracture, galling, fretting and attack by bodily fluids. A similar advance in the area of fixation stability will address the other major source of implant failure and would represent a significant advance in implant service life.

In cases of extreme loading conditions as is often the case for artificial hips, prosthetic joints may be made from metal alloys such as titanium, zirconium, or cobalt chrome alloys. Not only are these metal alloys of sufficient strength to withstand relatively extreme loading conditions, but due to their metallic nature, a metallic porous coating typically of Ti-6Al-4V may be secured to the metal alloy by a metallic bond. Such metallic porous coatings are useful for providing initial fixation of the implant immediately after surgery, but also serve to facilitate long-term stability by enhancing bone ingrowth and ongrowth.

While medical implant devices made from biocompatible metal alloys are effective, they may lack certain desirable characteristics. For example, metal alloys have poor flexibility and therefore do not tend to distribute load as evenly would be desired. Uneven loads tend to result in a gradual loosening of the implant. As such loosening becomes more sever, revision or replacement becomes necessary. For this reason, it is desirable to design medical implants generally and prosthetic joints specifically in such a way as to improve their in vivo fixation stability.

One way this problem has historically been addressed in the past is through the use of modified surfaces for medical implants which increase surface contact area and promote bone ingrowth and ongrowth. Another more recent technique involves the use of depositing material onto the surface of an implant, the material being the emission of a plasma spray source. This is discussed in U.S. Pat. Nos. 5,807,407 and 6,582,470, among others, which are incorporated by reference as though fully disclosed herein. In the present invention, we describe a unique and novel medical implant combining the advantages of oxidized zirconium with the advantages of a porous textured surface obtained through the use of plasma spray. The result is a medical implant which resists both articulative failure and fixation failure.

### BRIEF SUMMARY OF THE INVENTION

The present invention is directed to a medical implant and a method of making a medical implant. The implant comprises a surface of oxidized zirconium and a surface having material applied by plasma spray.

In some embodiments, of the present invention, there is a medical implant comprising a first component having a bearing surface; a second component having a counter-bearing surface adapted to cooperate with the bearing surface; wherein at least one of said first component or said second component comprises zirconium or zirconium alloy; a surface of oxidized zirconium on at least a portion of said first component, on at least a portion of said second component, or on at least a portion of both said first component and said second component; and, a porous, plasma sprayed coating on at least a portion of said surface of oxidized zirconium. In some embodiments, the medical implant is a joint prosthesis. In some embodiments of the joint prosthesis, the first component comprises a femoral component and the second component comprises an acetabular cup component to form a hip implant. In some embodiments of the joint prosthesis, the first component comprises a femoral component which further comprises at least one condyle and the second component comprises a tibial component to form a knee implant. In some embodiments of the joint prosthesis, the joint prosthesis is selected from the group consisting of shoulder, ankle, finger, wrist, toe, or elbow implants. In some embodiments of the medical implant, the medical implant is a maxillofacial or temporomandibular implant. In some embodiments of the medical implant, at least one of the first component or the second component comprises a metal selected from the group consisting of titanium, vanadium, hafnium, niobium, tantalum, cobalt, chromium, alloys thereof, and any combination thereof. In some embodiments of the medical implant, the porous, plasma sprayed coating comprises metal. In some embodiments having a porous, plasma sprayed coating, the porous, plasma sprayed coating comprises zirconium or zirconium alloy. In some embodiments having a porous, plasma sprayed coating comprising zirconium or zirconium alloy, the porous, plasma sprayed coating is oxidized to oxidized zirconium. In some embodiments of the medical implant having a porous, plasma sprayed coating, the porous, plasma sprayed coating comprises a metal selected from the group consisting of titanium, vanadium, hafnium, niobium, tantalum, cobalt, chromium, alloys thereof, and any combination thereof.

In some embodiments of the present invention, there is a medical implant comprising at least one component comprising a substrate comprising zirconium or zirconium alloy; a surface of oxidized zirconium on at least a portion of the component; and, a porous, plasma sprayed coating on at least a portion of the surface of oxidized zirconium. In some embodiments of the medical implant, the porous, plasma sprayed coating comprises metal. In some embodiments of the medical implant, the porous, plasma sprayed coating comprises zirconium or zirconium alloy. In some embodiments where the porous, plasma sprayed coating comprises zirconium or zirconium alloy, the porous, plasma sprayed coating is oxidized to oxidized zirconium. In some embodiments, the porous, plasma sprayed coating comprises a material selected from the group consisting of titanium, vanadium, hafnium, niobium, tantalum, cobalt, chromium, alloys thereof, and any combination thereof. In some embodiments of the medical implant the substrate comprises a metal selected from the group consisting of titanium, vanadium, hafnium, niobium, tantalum, cobalt, chromium, alloys thereof, and any combination thereof. In some embodiments of the medical implant, the medical implant is a vertebral implant. In some embodiments of the medical implant, the medical implant is a dental implant. In some embodiments of the medical implant, the medical implant comprises bone implant hardware. In some embodiments of the medical implant, the medical implant is bone implant hardware which comprises a bone plate or a bone screw.

In some embodiments of the present invention, there is a medical implant comprising a first component having a bearing surface; a second component having a counter-bearing surface adapted to cooperate with the bearing surface; wherein at least one of the first component or the second component comprises zirconium or zirconium alloy; a surface of oxidized zirconium on at least a portion of the first component, on at least a portion of the second component, or on at least a portion of both the first component and the second component; and, a porous, plasma sprayed coating on at least a portion of the first component or the second component or both the first component and the second component.

In some embodiments of the present invention, there is a method for manufacturing a medical implant having an oxidized zirconium surface and a porous coating comprising the steps of forming a medical implant wherein at least a portion of the medical implant comprises zirconium or zirconium alloy, applying a porous coating to at least a portion of the medical implant by plasma spray; and, oxidizing the medical implant comprising a zirconium or zirconium alloy to form a surface of oxidized zirconium. In some embodiments of the method, the medical implant further,comprises a material selected from the group consisting of titanium, vanadium, hafnium, niobium, tantalum, cobalt, chromium, alloys thereof, and any combination thereof. In some embodiments of the method, the step of oxidizing is performed before said step of applying. In some embodiments of the method, the plasma spray comprises a material selected from the group consisting of zirconium, titanium, vanadium, hafnium, niobium, tantalum, cobalt, chromium, alloys thereof, and any combination thereof.

In some embodiments of the present invention, there is a method for manufacturing a medical implant having a porous oxidized zirconium surface comprising the steps of forming the medical implant; applying a porous coating comprising zirconium or zirconium alloy to at least a portion of the medical implant by plasma spraying a material comprising zirconium or zirconium alloy; and, oxidizing the porous coating of zirconium or zirconium alloy to form a surface of oxidized zirconium. In some embodiments of the method, the medical implant comprises zirconium or zirconium alloy. In some embodiments of the method, the medical implant comprises a material selected from the group consisting of titanium, vanadium, hafnium, niobium, tantalum, cobalt, chromium, alloys thereof, and any combination thereof. In some embodiments of the method, the plasma sprayed coating comprising zirconium or zirconium alloy further comprises a material selected from the group consisting of titanium, vanadium, hafnium, niobium, tantalum, cobalt, chromium, alloys thereof, or any combination thereof.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated that the conception and specific embodiment disclosed may be readily utilized as a basis for modifying or designing other structures for carrying out the same purposes of the present invention. It should also be realized that such equivalent constructions do not depart from the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIG. 1 is a schematic of a typical hip implant.

FIG. 2 is a schematic of a typical knee implant.

FIG. 3 is a schematic of the type of apparatus that may be used for the plasma spray of medical implants.

FIG. 4 is a flow diagram of the process steps in the plasma spray of medical implants.

### DETAILED DESCRIPTION OF THE INVENTION

As used herein, “a” or “an” means one or more. The singular includes the plural and the plural includes the singular.

As used herein, the term alloy is defined broadly such that “an alloy of metal x” encompasses alloys having any amount of x, and does not require that metal x be present as either the single most common component or that it be present at some minimum level. Thus, an alloy qualifies as “an alloy of metal x” even if metal x is present at low levels such as 1% or less.

As used herein, the term “metal”, “metallic”, or “metallic material” includes a pure metal or metal alloy.

The invention provides, in part, oxidized zirconium coated orthopedic implants or prostheses fabricated of zirconium or zirconium containing metal alloys or a thin coating of zirconium or zirconium alloy on conventional orthopedic implant materials. The oxidized zirconium herein described throughout is the blue-black or black oxidized zirconium described by Davidson in U.S. Pat. No. 5,037,438 and by Watson in U.S. Pat. No. 2,987,352, both of which are incorporated by reference as though fully described herein. In order to form continuous and useful oxidized zirconium coatings over the desired surface of the metal alloy prosthesis substrate, the metal alloy preferably contain from about 80 to about 100 wt % zirconium, most preferably from about 95 to about 100 wt %. Oxygen, niobium, and titanium include common alloying elements in the alloy with often times the presence of hafhium. Yttrium may also be alloyed with the zirconium to enhance the formation of a tougher, yttria-stabilized oxidized zirconium coating during the oxidation of the alloy. While such zirconium containing alloys may be custom formulated by conventional methods known in the art of metallurgy, a number of suitable alloys are commercially available. These commercial alloys include among others Zircadyne 705, Zircadyne 702, and Zircalloy.

The base zirconium containing metal alloys are cast or machined by conventional methods to the shape and size desired to obtain a prosthesis substrate. The substrate is then subjected to process conditions which cause the natural (in situ) formation of a tightly adhered, diffusion-bonded coating of oxidized zirconium on its surface. The process conditions include, for instance, air, steam, or water oxidation or oxidation in a salt bath. These processes ideally provide a thin, hard, dense, blue-black or black, low-friction wear-resistant oxidized zirconium film or coating of thicknesses typically on the order of several microns (10⁶ meters) on the surface of the prosthesis substrate. Below this coating, diffused oxygen from the oxidation process increases the hardness and strength of the underlying substrate metal.

The air, steam and water oxidation processes and the oxidized zirconium surfaces (blue-black or black oxidized zirconium) produced therefrom are described in now-expired U.S. Pat. No. 2,987,352 to Watson and in U.S. Pat. No. 5,037,438 to Davidson, the teachings of which are incorporated by reference as though fully set forth. The air oxidation process provides a firmly adherent black or blue-black layer of oxidized zirconium of highly oriented monoclinic crystalline form. If the oxidation process is continued to excess, the coating will whiten and separate from the metal substrate. The oxidation step may be conducted in either air, steam or hot water. For convenience, the metal prosthesis substrate may be placed in a furnace having an oxygen-containing atmosphere (such as air) and typically heated at 700° F.-1100° F. up to about 6 hours. However, other combinations of temperature and time are possible. When higher temperatures are employed, the oxidation time should be reduced to avoid the formation of the white oxide.

It is preferred that a blue-black oxidized zirconium layer ranging in thickness from about 1 to about 20 microns should be formed. Most preferably, the thickness should be from about 1 to 5 microns. For example, furnace air oxidation at 1000° F. for 3 hours will form an oxide coating on Zircadyne 705 about 4-5 microns thick. Longer oxidation times and higher oxidation temperatures will increase this thickness, but may compromise coating integrity. For example, one hour at 1300° F. will form an oxide coating about 14 microns in thickness, while 21 hours at 1000° F. will form an oxide coating thickness of about 9 microns. Of course, because only a thin oxide is necessary on the surface, only very small dimensional changes, typically less than 10 microns over the thickness of the prosthesis, will result. In general, thinner coatings (1-4 microns) have better attachment strength.

One of the salt-bath methods that may be used to apply the oxidized zirconium coatings to the metal alloy prosthesis, is the method of U.S. Pat. No. 4,671,824 to Haygarth, the teachings of which are incorporated by reference as though fully set forth. The salt-bath method provides a similar, slightly more abrasion resistant blue-black or black oxidized zirconium coating. The method requires the presence of an oxidation compound capable of oxidizing zirconium in a molten salt bath. The molten salts include chlorides, nitrates, cyanides, and the like. The oxidation compound, sodium carbonate, is present in small quantities, up to about 5 wt. %. The addition of sodium carbonate lowers the melting point of the salt. As in air oxidation, the rate of oxidation is proportional to the temperature of the molten salt bath and the '824 patent prefers the range 550° C.-800° C. (1022° F.-1470° F.). However, the lower oxygen levels in the bath produce thinner coatings than for furnace air oxidation at the same time and temperature. A salt bath treatment at 1290° F. for four hours produces an oxide coating thickness of roughly 7 microns.

Whether air oxidation in a furnace or salt bath oxidation is used, the oxidized zirconium coatings are quite similar in hardness. For example, if the surface of a wrought Zircadyne 705 (Zr, 2-3 wt % Nb) prosthesis substrate is oxidized, the hardness of the surface shows a dramatic increase over the 200 Knoop hardness of the original metal surface. The surface hardness of the blue-black oxidized zirconium surface following oxidation by either the salt bath or air oxidation process is approximately 1700-2000 Knoop hardness.

These diffusion-bonded, low friction, highly wear resistant oxidized zirconium coatings are applied to the surfaces of orthopedic implants subject to conditions of wear. Such surfaces include the articulating surfaces of knee joints, elbows and hip joints. As mentioned before, in the case of hip joints, the femoral head and stem are typically fabricated of metal alloys while the acetabular cup may be fabricated from ceramics, metals or organic polymer-lined metals or ceramics.

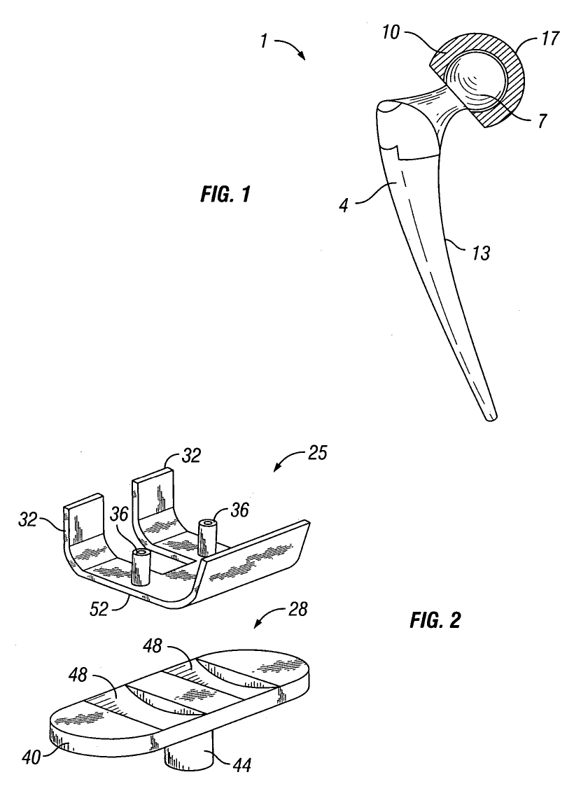

FIG. 1 illustrates a typical hip joint assembly (1). The hip joint stem (4) fits into the femur and contacts the femur through it outer surface (13) while the femoral head (7) of the prosthesis fits into and articulates against the inner lining of an acetabular cup (10) which is affixed to the pelvis and contacts the pelvis through its outer surface (17). This allows the fabrication of a prosthesis having a metallic stem and neck but a femoral head of some other material, such as ceramic. Materials are chosen, primarily, to enhance the useful life of the implant. The development of the use of oxidized zirconium surfaces has considerably lengthened the useful life of implants. However, another mode of failure results from the loss of fixation of the implant in the implant site. Over time, the fixation stability of typical implants deteriorates. A major contributing factor in this deterioration is the loosening of the components where they contact tissue. To this end, oxidized zirconium-based implants can be combined with the plasma spray deposition of material onto the implant surface. Such a modified surface improves fixation by increasing the contact surface area and promoting bone ingrowth and ongrowth to the implant. Material may be applied by plasma spray onto some or all of the surfaces of the implant. In the case of the hip prosthesis described above, it can be advantageously applied to the surface of the hip stem (13) or the surface of the acetabular cup (17), as well as other areas.

A typical knee joint prosthesis is shown in situ in FIG. 2. The knee joint includes a femoral component (25) and a tibial component (28). The femoral component includes condyles (32) which provide the articulating surface of the femoral component and pegs (36) for affixing the femoral component to the femur. The tibial component (28) comprises a tibial platform (40) which is supplied with grooves (48) similar to the shape of the condyles (32). The bottom surfaces of the condyles (52) contact the tibial platform's grooves (48) so that the condyles articulate within these grooves against the tibial platform. As with the hip prosthesis, material may be applied by plasma spray onto some or all of the surfaces of the implant to improve fixation. Preferably, application of material by plasma spray is performed on those surfaces that directly contatct tissue. Modifying the surfaces which contact tissue in this way improves fixation by promoting bone ingrowth and ongrowth to the implant.

When the oxidized zirconium coated femoral head is used in conjunction with any of these acetabular cups, the coefficient of friction between the femoral head and the inner surface of the cup is reduced so that less heat and torque is generated and less wear of the mating bearing surface results. This reduction in heat generation, frictional torque, and wear is particularly important in the case of acetabular cups lined with organic polymers or composites of such polymers. Organic polymers, such as UHMWPE, exhibit rapidly increased rates of creep when subjected to heat with consequent deleterious effect on the life span of the liner. Wear debris of the polymer leads to adverse tissue response and loosening of the device. Thus, not only does the oxidized zirconium coating serve to protect the prosthesis substrate to which it is applied and increase its mechanical strength properties but, as a result of its low friction surface, it also protects those surfaces against which it is in operable contact and consequently enhances the performance and life of the prosthesis.

The usefulness of oxidized zirconium coated prosthesis is not limited to load bearing prostheses, especially joints, where a high rate of wear may be encountered. Because the oxidized zirconium coating is firmly bonded to the zirconium alloy prosthesis substrate, it provides a barrier between the body fluids and the zirconium alloy metal thereby preventing the corrosion of the alloy by the process of ionization and its associated metal ion release.

Oxygen diffusion into the metal substrate during oxidation also increases the strength of the metal. Consequently, an oxidized zirconium coated prosthesis may be expected to have a greater useful service life.

Zirconium or zirconium alloy can also be used to provide a porous bead or wire mesh surface to which surrounding bone or other tissue may integrate to stabilize the prosthesis. These porous coatings can be treated simultaneously by the oxidation treatment in a manner similar to the oxidation of the base prosthesis for the elimination or reduction of metal ion release. Furthermore, zirconium or zirconium alloy can also be used as a surface layer applied over conventional implant materials prior to in situ oxidation and formation of the oxidized zirconium coating.

The combination of oxidized zirconium-based implants and textured, porous surfaces using plasma spray technology results in a medical implant having heretofore unparalleled service life. In fabricating medical implants of oxidized zirconium, it is preferred that the unique and beneficial properties of the material is not compromised by conditions of the manufacturing process. For example, changes in the underlying substrate as well as changes in the oxidized zirconium coating itself are not preferred. It has been found that the application of a plasma sprayed coating to oxidized zirconium surfaces does not have deleterious effects on either the surface or on the microstructure of the substrate.

The introduction of a textured, porous surface through the use of plasma spraying of a metal has been used in the construction of medical implants. Typically, this has been pure titanium on the surface of an implant, such as one of Ti-6Al-4V. Alternatively, a zirconium or zirconium alloy, such as those useful in the formation of oxidized zirconium surfaces may be used. However, other metals may be used, including, but not limited to, titanium, vanadium, hafnium, niobium, tantalum, cobalt, chromium, alloys thereof, and any combination thereof. In one example of this technique, the spraying parameters are adjusted such that the metal powder or wire being injected into the plasma is only partially melted as it is being accelerated toward the substrate. Plasma sprayed coatings, like sintered coatings, are 500 to 1000 microns thick, but they do form a regular three-dimensional interconnected array of pores. The plasma sprayed coatings essentially form irregular surfaces with very little interconnected porosity throughout the thickness of the coating. Bone tissue will integrate onto the textured coating, providing enhanced implant fixation.

A plasma is a gas (or cloud) of charged and neutral particles exhibiting collective behavior which is formed by excitation of a source gas or vapor. A reactive plasma generates many chemically active charged (ionic) and neutral (radical) species. These charged and neutral species are more active etchants than the original source gas. The reaction of the active neutral and ionic species on the surface of a work piece etches, or removes, portions of the surface. The physical striking of ions on the surface of the implant work piece (i.e., sputtering) further enhances the etch rate. Thus in a “reactive plasma” process, both physical sputtering of ions and dry chemical etching by the active neutral and ionic species occurs. Not all of the surface atoms liberated by sputtering or reactive etch are immediately removed from the system and, if conditions are favorable, considerable redeposition of the atoms of the titanium surface may occur. Further, the different atoms present on the exposed surface will undergo reactive chemical etching and physical sputtering at different rates. Thus, the etch rate and redeposition rate may be controlled by controlling the reactive plasma feed composition, work piece composition, gas pressure, plasma power, voltage bias across the sheath, reactor geometry, workpiece dimension and process time.

For application to medical implant devices, it is desirable that the mode of etching does not introduce further contaminants onto the surface of the work piece. Thus, according to the invention, a fluorine- or chlorine-containing source gas may be used to generate an active etching chlorine or fluorine species. The active species may be a radical or charged species. Although iodine-containing gases have not been used to etch titanium, they may be useful to treat other metal surfaces. Titanium reacts with the active etchant species of Cl and F radicals to form a volatile titanium halide which is pumped away by the system. For example, TiCl₄ has an evaporation temperature of 135° C.; and TiF₄ has a sublimation temperature of 285° C. at atmospheric pressure. Under low pressure operating conditions, these volatile halides may evaporate at ambient temperatures. Thus, no impurities are introduced into the work piece by the etching process, as all by-products are rapidly removed from the work piece surface.

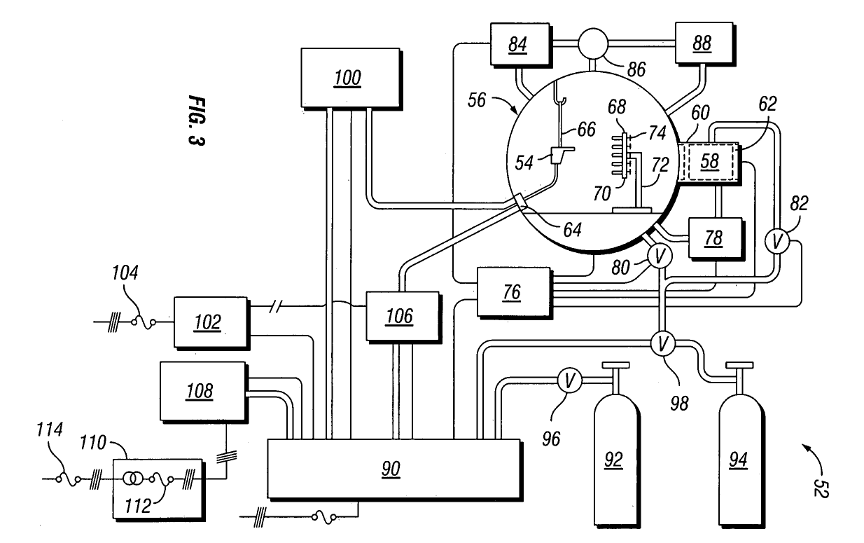

An illustrative, non-limiting example of an apparatus 52 for applying the porous coating to the structural member will now be described with reference to FIG. 3. It should be understood that any method of plasma spray deposition is useful in the present invention. The apparatus 52 includes a plasma spray gun 54 which is disposed within a spray chamber 56. The plasma spray gun 54 receives a controlled mixture of helium and argon as well as a powdered material such as Ti-6Al-4V, pure titanium, zirconium, or zirconium alloy which is used to form the porous coating. The plasma spray gun 54 ionizes the mixture of helium and argon to form a plasma which has a temperature of approximately 17,000° F. by creating a relatively large voltage differential between the anode and cathode of the plasma spray gun 54. The powdered material which forms the porous coating is then injected at a point just in front of the plasma leaving the plasma spray gun 54 such that melted particles of the powdered material melt a portion of the structural member and become partially embedded on the outer surface of the structural member. The melted particles leaving the plasma spray gun 54 have a velocity of between 400 and 600 meters per second. The plasma spray gun 54 may be any suitable plasma spray gun. These devices are known in the art.

The spray chamber 56 includes a pass-through chamber 58 having an internal door 60 and an external door 62. The external door 62 is used to allow the medical implant device to be placed within the pass-through chamber 58 and then permits the pass-through chamber 58 to be sealed from the environment while the medical implant device is removed through the inner door 60 and placed on a fixture described below. The spray chamber 56 further includes a plasma hook-up feed 64 which is used to deliver power, the powdered material, and gases to the plasma spray gun 54. In addition, the spray chamber 56 further includes a counterbalancing device 66 which is secured to the plasma spray gun 54 and allows the plasma spray gun 54 to be manipulated by the operator without causing the weight of the plasma spray gun 54 to fatigue the operator. The spray chamber 56 may be any suitable spray chamber.

Disposed within the spray chamber 56 is a fixture 42 for supporting a plurality of medical implant devices 10 within the spray chamber 30. The fixture 68 includes an annular member 70 which is used to receive approximately six medical implant devices. The annular member 70 is able to rotate with respect to a support member 72 so as to allow the operator of the plasma spray gun to have relatively easy access to each of the medical implant devices. In addition, the annular member 70 includes a plurality of knobs 74 which permit rotation of each of the medical implant devices individually with respect to the annular member 70 so as to allow all surfaces of each medical implant device to be accessible to the operator of the plasma spray gun 54.

To control the operation of the spray chamber 56, the apparatus 52 further includes a chamber controller 76. The chamber controller 76 is used to control the evacuation of the spray chamber 56 and the pass-through chamber 58 by means of a vacuum pump 78. The vacuum pump 78 has the capacity to reduce the pressure within the spray chamber 56 as well as the pass-through chamber 58 to approximately 10 millitorr. In addition, the chamber controller 76 is able to control the pressure in the spray chamber 56 and the pass-through chamber 58 during the plasma spray operation through the valves 80 and 82. The chamber controller 76 is connected to a control unit which is more fully described below. Any suitable chamber controller and vacuum pump may be used.

As will be appreciated by those skilled in the art, the spray chamber 56 becomes hot during the plasma spray operation. To remove heat from the spray chamber 56 during this operation, the apparatus 52 further includes a chamber blower 84, a tube cooler 86, as well as a chamber chiller 88. The chamber blower 84 is controlled by the chamber controller 76 and is used to receive heated argon and helium from the spray chamber 56 and deliver the gas to the tube cooler 86. The tube cooler 86 has a plurality of half-inch diameter copper tubes having ethylene glycol flowing therethrough which act as a heat exchanger. The chamber chiller 88 is in turn used to cool the ethylene glycol flowing in the tube cooler 86 to approximately 0° C. The argon and helium which has passed through the tube cooler 86 is then reintroduced into the spray chamber 56. Any suitable chillers and tube access may be used.

The apparatus further includes a control unit 90 which is used to control the flow of argon and helium as well as control the current which is delivered to the plasma spray gun 54. The control unit 90 also serves to control the speed at which the powder is delivered to the plasma spray gun 54 as more fully described below, as well as monitors the temperature at which the plasma spray gun 54 operates by recording the temperature of the water leaving the plasma spray gun 54. In addition, the control unit 90 determines whether the pressure of argon gas delivered to the plasma spray gun 54 is within a predetermined range. If either the pressure of the ionizing gases delivered to the plasma spray gun 54 falls outside the predetermined range or the temperature of the water from the plasma spray gun 54 becomes too high (e.g., exceeds 35° C.), the control unit 90 terminates the plasma spray operation until these conditions are corrected. The control unit 90 also monitors the ratio of argon to helium delivered to the spray chamber 56 during the plasma spray operation so as to maintain a ratio of 30 standard liters of argon to 10 standard liters of helium. Any suitable control unit may be used.

The control unit 90 receives helium gas from a first tank 92 through a first regulator diaphragm valve 96 as well as argon gas through the second regulator diaphragm valve 98. The second regulator diaphragm valve 98 also controls the flow of argon gas to the electronically actuated valves 80 and 82 which in turn controls the flow of argon gas to the spray chamber 56 as well as the pass-through chamber 58. Any suitable control unit may be used.

To deliver powdered material which is used to form the porous coating to the plasma spray gun 54, the apparatus 52 further includes a powder feeder 100. The powder feeder 100 is controlled by the control unit 90 and may be used to simultaneously deliver two grades of powdered metal to the plasma spray gun 54. For illustrative purposes, the first powder may be a fine grade of 80-200 mesh, while the second powder may be a coarse grade of 60-100 mesh. Both the fine and coarse grades of powder are delivered to the plasma spray gun 54 by a flow of argon gas which is delivered from the control unit 90. The flow rate of argon gas for the fine grade powder is 2.3 liters/minute while the flow rate for the course grade is 2.6 liters/minute. The wheel speed associated with the powder feeder 100 is 16% of the maximum wheel speed for the fine grade powder, while the wheel speed associated with the course grade powder is 15% of the maximum. However, it will be understood that the flow rate of the gas, current and powder feed rates may be those valves which are recommended for the particular type of the powder which is used. Any suitable powder feeder may be used.

To supply power for operating the control unit 90 as well as the plasma spray gun 54, the apparatus 52 further includes a first power supply 102. The first power supply 102 receives electrical energy from a source (not shown) through a slow burning 300 amp fuse 104. The first power supply 102 is able to generate 105 kVA and is used to supply the control unit 90 as well as the plasma spray gun 54 through a jam box 106. Any suitable power supply may be used.

The apparatus 52 further may include a freon chiller 108 which is used to cool the water which circulates through the plasma spray gun 54. The freon chiller 108 is controlled by the control unit 90 and delivers cooling water to the control unit 90 which in turn is delivered to the plasma spray gun 54. In this regard, the freon chiller 108 preferably cools the water entering the plasma spray gun 54 to approximately 17° C. Electrically communicating with the freon chiller 108 is a second power supply 110 which is used to provide a regulated supply of voltage to the freon chiller 108. The second power supply 110 may include a 35 amp slow burning fuse 112 and receives power from a source (not shown) through a 60 amp slow burning fuse 114. Any suitable freon chiller may be used.

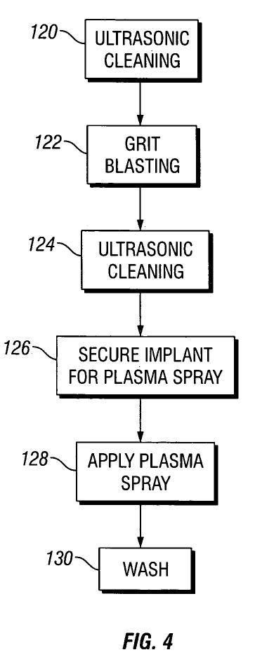

An example of the operation of the apparatus 52 will now be described with reference to FIG. 4. Prior to placing the structural member in the spray chamber 56, the structural member is first ultrasonically cleaned in water to remove surface contaminants as indicated by the step 120. Following the first ultrasonic cleaning step 120, the structural member is subjected to a grit blasting operation as represented by the step 122. In this regard, the areas of the structural member which are to be free of the porous coating is initially covered with polyvinyl chloride tape. The structural member is then placed in front of the grit blaster operating at 40 psi with a half-inch nozzle and using 16 grit silicon carbide particles. By grit blasting the structural member, a roughened surface is formed in the region which is exposed to the particles.

The polyvinyl chloride tape is removed and the structural member is then cleaned in a second ultrasonic cleaning operation as represented by the step 124. The portions of the structural member which are not to be covered by the porous coating are then covered by heat tape which is resistant to the high temperatures which are generated during the plasma spraying operation. Care should be taken so that the portions of the structural member which are to be covered by the porous coating are not physically contacted after the second ultrasonic cleaning step 124 until after the porous coating has been applied. In this regard, the storage racks which are used to transport the structural member may support the structural member only at those areas which are not to be coated with the porous coating.

The spray chamber 56 is initially evacuated to approximately 10 millitorr by the vacuum pump 78 and then backfilled with argon gas to a pressure slightly above atmospheric. The structural member is then placed in the pass-through chamber 32 through the exterior door 36 and then the pass-through chamber 58 is closed. The operator of the spray chamber 56 then uses the gas-impermeable arm length rubber gloves within the spray chamber 56 to open the inner door 60 of the pass-through chamber 58, remove the structural member and secure the structural member to the fixture 68 as indicated by the step 124. Again, care is taken to make sure that no contact is made with the portions of the structural member which are to receive the porous coating.

At steps 126 and 128, the operator of the spray chamber 56 directs the plasma spray gun 54 at one of the structural members on the fixture 68 while the structural member is rotated by using one of the knobs 54. The plasma spray is then applied to the structural member for a period of approximately 20 seconds after which the annular member 70 is rotated in such a manner as to place the structural member to which the plasma spray has been applied proximate to the exhaust of the tube cooler 86 for approximately one minute. By placing the structural member in front of the exhaust of the tube cooler 86 immediately after receiving the plasma spray, the thermal energy within the structural member is able to dissipate a sufficient amount so as to prevent the structural member from melting.

While one of the structural members is in front of the exhaust of the tube cooler 86, the operator may begin the process of applying the porous coating on to another structural member in the same manner as described above. This process continues until each of the structural members on the fixture 42 has received one application of plasma spray. Once each of the structural members has received one application of plasma spray, the process is repeated until eight coatings have been applied to each of the structural members. After each of the structural members have received eight applications of plasma spray, each of the structural members with the porous coating are removed from the spray chamber 56 through the pass-through chamber 58 and then each of the medical implant devices are sandpapered to remove loose material and then are washed in a water jet operating at 900 psi to remove additional residue as indicated by step 130.

The present invention seeks to combine the unique advantages of oxidized zirconium-based medical implants and porous, textured surfaces which can be achieved through plasma spray application of material. The use of oxidized zirconium surfaces on medical implants has realized significant gains in the service life of such implants due to its unparalleled combination of strength, low surface roughness, and good thermal properties. Combining such implants with the improved fixation properties of a porous textured surface would address the remaining major source of implant failure, namely loosening of the implant in the implant site.

The present invention includes the application of plasma spray to a medical implant with having, at least in part, an oxidized zirconium surface. The material so applied by plasma spray could be zirconium or zirconium alloy, but it could also be another metal or metal alloy. Alternatively, the metallic material applied by plasma spray may also be a zirconium or zirconium alloy which is then oxidized to oxidized zirconium. Preferably, the implant has articulating surfaces comprising oxidized zirconium and porous, textured fixation surfaces produced by plasma spray. Preferably, the plasma sprayed fixation surface is that of titanium or a titanium alloy such as Ti-6Al-4V, but may be a zirconium or zirconium alloy. If it is zirconium or zirconium alloy, the material so deposited may be oxidized to form a porous, textured surface of oxidized zirconium.

In the most general embodiment, the medical implant comprises a substrate of zirconium or zirconium alloy. Preferably, the medical implant is a multi-component joint prosthesis having bearing and counter-bearing surfaces which articulate against one another, such as a hip or knee prosthesis. Other prostheses such as shoulder, ankle, finger, wrist, toe, elbow and others are useful in the present invention. Maxillofacial or temporomandibular implants are other examples. Preferably, the components are made from any acceptable material for a medical implant, including, but not limited to, zirconium, titanium, vanadium, hafnium, niobium, tantalum, cobalt, chromium, alloys thereof, and any combination thereof. Preferably, the plasma sprayed material is a metal. Most preferably, it comprises zirconium or zirconium alloy. In some cases, the plasma sprayed zirconium or zirconium alloy is oxidized to oxidized zirconium. The plasma sprayed material can also comprise other metals, including but not limited to titanium, vanadium, hafnium, niobium, tantalum, cobalt, chromium, alloys thereof, and any combination thereof. The plasma sprayed material resides on at least a part of the surface of oxidized zirconium. In the more general embodiment, the medical implant may be a vertebral implant, a dental implant, or other implant. It may also be bone implant hardware such as bone plates, bone screws, or other known examples of implant hardware.

Alternatively, the medical implant is a multi-component joint prosthesis having bearing and counter-bearing surfaces which articulate against one another, similar to that described above, but differing in that the plasma sprayed surface need not necessarily reside atop the surface of oxidized zirconium. For example, the oxidized zirconium surfaces may be limited to those surface which articulate against one another or those which otherwise are subject to enhanced wear, while the plasma sprayed porous surface resides elsewhere on the implant to enhance tissue ingrowth.

In the manufacturing the medical implant, the formation of the oxidized zirconium surface is preferably performed by oxidation prior to the application of a plasma spray coating. However, in some cases, this may be reversed. The oxidized zirconium may be formed by oxidation of zirconium or zirconium alloy in the substrate or it may be formed by oxidation of plasma sprayed zirconium or zirconium alloy. In those cases where the plasma sprayed material is a zirconium or zirconium alloy which is to be oxidized to oxidized zirconium, at least one oxidation step must occur after the step of plasma spraying the implant.

Although the present invention and its advantages have been described in detail, it should be understood that various changes, substitutions and alterations can be made herein without departing from the invention as defined by the appended claims. Moreover, the scope of the present application is not intended to be limited to the particular embodiments of the process, machine, manufacture, composition of matter, means, methods and steps described in the specification. As one will readily appreciate from the disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

What is claimed is:

1. A medical implant comprising:
a first component having a bearing surface;
a second component having a counter-bearing surface adapted to cooperate with the bearing surface;
wherein at least one of said first component or said second component comprises zirconium or zirconium alloy;
a surface of oxidized zirconium on at least a portion of said first component, on at least a portion of said second component, or on at least a portion of both said first component and said second component; and,
a porous, plasma sprayed coating on at least a portion of said surface of oxidized zirconium.

2. The medical implant of claim 1, wherein said medical implant is a joint prosthesis.

3. The medical implant of claim 2, wherein said first component comprises a femoral component and said second component comprises an acetabular cup component to form a hip implant.

4. The medical implant of claim 2, wherein said first component comprises a femoral component which further comprises at least one condyle and said second component comprises a tibial component to form a knee implant.

5. The medical implant of claim 2, wherein said joint prosthesis is selected from the group consisting of shoulder, ankle, finger, wrist, toe, or elbow implants.

6. The medical implant of claim 1, wherein said medical implant is a maxillofacial or temporomandibular implant.

7. The medical implant of claim 1, wherein at least one of said first component or said second component comprises a metal selected from the group consisting of titanium, vanadium, hafnium, niobium, tantalum, cobalt, chromium, alloys thereof, and any combination thereof.

8. The medical implant of claim 1, wherein said porous, plasma sprayed coating comprises metal.

9. The medical implant of claim 8, wherein said porous, plasma sprayed coating comprises zirconium or zirconium alloy.

10. The medical implant of claim 9, wherein said porous, plasma sprayed coating is oxidized to oxidized zirconium.

11. The medical implant of claim 8, wherein said porous, plasma sprayed coating comprises a metal selected from the group consisting of titanium, vanadium, hafnium, niobium, tantalum, cobalt, chromium, alloys thereof, and any combination thereof.

12. A medical implant comprising:
at least one component comprising:
a substrate comprising zirconium or zirconium alloy;
a surface of oxidized zirconium on at least a portion of said component; and,
a porous, plasma sprayed coating on at least a portion of said surface of oxidized zirconium.

13. The medical implant of claim 12, wherein said porous, plasma sprayed coating comprises metal.

14. The medical implant of claim 13, wherein said porous, plasma sprayed coating comprises zirconium or zirconium alloy.

15. The medical implant of claim 14, wherein said porous, plasma sprayed coating is oxidized to oxidized zirconium.

16. The medical implant of claim 13, wherein said porous, plasma sprayed coating comprises a material selected from the group consisting of titanium, vanadium, hafnium, niobium, tantalum, cobalt, chromium, alloys thereof, and any combination thereof.

17. The medical implant of claim 12, wherein said substrate comprises a metal selected from the group consisting of titanium, vanadium, hafnium, niobium, tantalum, cobalt, chromium, alloys thereof, and any combination thereof.

18. The medical implant of claim 12, wherein said medical implant is a vertebral implant.

19. The medical implant of claim 12, wherein said medical implant is a dental implant.

20. The medical implant of claim 12, wherein said medical implant comprises bone implant hardware.

21. The medical implant of claim 12, wherein said bone implant hardware comprises a bone plate or a bone screw.

22. A medical implant comprising:
a first component having a bearing surface;
a second component having a counter-bearing surface adapted to cooperate with the bearing surface;
wherein at least one of said first component or said second component comprises zirconium or zirconium alloy;
a surface of oxidized zirconium on at least a portion of said first component, on at least a portion of said second component, or on at least a portion of both said first component and said second component; and,
a porous, plasma sprayed coating on at least a portion of said first component or said second component or both said first component and said second component.

23. A method for manufacturing a medical implant having an oxidized zirconium surface and a porous coating comprising the steps of:
forming a medical implant wherein at least a portion of said medical implant comprises zirconium or zirconium alloy,
applying a porous coating to at least a portion of said medical implant by plasma spray; and,
oxidizing said medical implant comprising a zirconium or zirconium alloy to form a surface of oxidized zirconium.

24. The method of claim 23, wherein said medical implant further comprises a material selected from the group consisting of titanium, vanadium, hafnium, niobium, tantalum, cobalt, chromium, alloys thereof, and any combination thereof.

25. The method of claim 23, wherein said step of oxidizing is performed before said step of applying.

26. The method of claim 23, wherein said plasma spray comprises a material selected from the group consisting of zirconium, titanium, vanadium, hafnium, niobium, tantalum, cobalt, chromium, alloys thereof, and any combination thereof.

27. A method for manufacturing a medical implant having a porous oxidized zirconium surface comprising the steps of:
forming said medical implant;
applying a porous coating comprising zirconium or zirconium alloy to at least a portion of said medical implant by plasma spraying a material comprising zirconium or zirconium alloy; and,
oxidizing said porous coating of zirconium or zirconium alloy to form a surface of oxidized zirconium.

28. The method of claim 27, wherein said medical implant comprises zirconium or zirconium alloy.

29. The method of claim 27, wherein said medical implant comprises a material selected from the group consisting of titanium, vanadium, hafnium, niobium, tantalum, cobalt, chromium, alloys thereof, and any combination thereof.

30. The method of claim 27, wherein said plasma sprayed coating comprising zirconium or zirconium alloy further comprises a material selected from the group consisting of titanium, vanadium, hafnium, niobium, tantalum, cobalt, chromium, alloys thereof, or any combination thereof.
